(19) United States
(12) Patent Application Publication
(10) Pub. No.: US 2007/0276020 A1
(43) Pub. Date: Nov. 29, 2007

(54) Dicationic Compounds For Activity Against Trichomonas Vaginalis

(76) Inventors: David W. Boykin, Atlanta, GA (US); Chad E. Stephens, Carrollton, GA (US); W. Evan Secor, Atlanta, GA (US); Andrea L. Crowell, Holland, OH (US); Arvind Kumar, Lilburn, GA (US)

Correspondence Address: JENKINS, WILSON, TAYLOR & HUNT, P. A., 3100 TOWER BLVD., Suite 1200, DURHAM, NC, 27707, US

(21) Appl. No.: 10/591,096

(22) PCT Filed: Mar. 7, 2005
(86) PCT No.: PCT/US05/07316

§ 371 Date: May 11, 2007

Related U.S. Application Data
(60) Provisional application No. 60/551,089, filed on Mar. 8, 2004.

(51) Int. Cl. A61K 31/341; A61K 31/4184; A61P 33/02; C07D 307/54; C07D 403/10; C07D 405/14; C07D 409/04

(52) U.S. Cl. 514/394; 514/471; 548/304.7; 548/305.4; 549/491

(57) ABSTRACT

Dicationic compounds for the treatment of T. vaginalis infections are described. The presently described compounds exhibit in vitro activity against metronidazole-sensitive and -resistant T. vaginalis isolates. Furthermore, the presently described compounds demonstrate IC₅₀ concentrations that were not elevated in the metronidazole resistant isolate, suggesting that their activity is not affected by parasite mechanisms that confer resistance to 5-nitroimidizoles.

### RELATED APPLICATIONS

This application claims the benefit of U.S. Provisional Patent Application Serial No. 60/551,089, filed Mar. 8, 2004; the disclosure of which is incorporated herein by reference in its entirety.

### GOVERNMENT INTEREST

The work was supported by the Office of Minority and Women's Health, National Center for Infectious Diseases, Centers for Disease Control and Prevention. The compound synthesis activities were supported by NIH grant No. NAID RO1AI46365. The U.S. government has certain rights in the invention.

### TECHNICAL FIELD

The presently disclosed subject matter relates to methods of treating trichomoniasis infections with novel dicationic compounds, processes of synthesizing novel dicationic compounds, and to the novel compounds themselves. More particularly, the presently disclosed subject matter relates to methods of treating infections caused by the protozoan parasite Trichomonas vaginalis with novel dicationic compounds.

ABBREVIATIONS

| | |
|---|---|
| δ^{- =} | chemical shift |
| Ac = | acetyl |
| AcO = | acetoxyl |
| AcOH = | acetic acid |
| Ac₂O = | acetic anhydride |
| Am = | amidine |
| AmOH = | amidoxime |
| Bn = | benzyl |
| Bu = | butyl |
| Bz = | benzoyl |
| ° C. = | degrees Celsius |
| calcd = | calculated |
| CDC = | Centers for Disease Control |
| CDCl₃ = | deuterated chloroform |
| cm = | centimeters |
| dec = | decomposition point |
| DIBAL = | diisobutylaluminium hydride |
| DMF = | dimethylformamide |
| DMSO = | dimethylsulfoxide |
| D₂O = | deuterium oxide |
| Et = | ethyl |
| Et₂O = | diethyl ether |
| EtOAc = | ethyl acetate |
| EtOH | ethanol |
| g = | grams |
| GC = | gas chromatography |
| GLC = | gas-liquid chromatography |
| h = | hours |
| HCl = | hydrogen chloride |
| HPLC = | high-pressure liquid chromatography |
| Hz = | hertz |
| ip = | intraperitoneal |
| IR = | infrared |
| kg = | kilograms |
| KO-t-Bu = | potassium tert-butoxide |
| M = | molar |
| Me = | methyl |
| MeO = | methoxyl |
| MHz = | megahertz |
| mL = | milliliters |
| MLC = | minimal lethal concentration |
| mm = | millimeters |
| mmol = | millimole |
| μM = | micromolar |
| m.p. = | melting point |
| MS = | mass spectroscopy |
| NaCl = | sodium chloride |
| NaHCO₃ = | sodium bicarbonate |
| Na₂CO₃ = | sodium carbonate |
| Na₂HPO₄ = | sodium hydrogen phosphate |
| Na₂SO₄ = | sodium sulfate |
| NaOH = | sodium hydroxide |
| NBS = | N-bromosuccinimide |
| NH₂OH·HCl = | hydroxylamine hydrochloride |
| NMR = | nuclear magnetic resonance |
| p = | para |
| Ph = | phenyl |
| Pd-C = | 10% palladium on carbon |
| psi = | pounds per square inch |
| po = | oral |
| spp. = | species |
| TBME = | tert-butyldimethyl ether |
| THF = | tetrahydrofuran |
| TLC = | thin-layer chromatography |
| TMS = | trimethylsilyl |
| T. vaginalis= | Trichomonas vaginalis |
| TYM = | trypticase-yeast-maltose medium |
| UV = | ultraviolet |

### BACKGROUND

Trichomoniasis is a common sexually transmitted disease caused by the protozoan parasite Trichomonas vaginalis. An estimated 170 million persons are infected with T. vaginalis worldwide. See World Health Organization, An Overview of Selected Curable Sexually Transmitted Diseases, in Global Program on AIDS (World Health Organization, Geneva, Switzerland), 2-27 (1995). Clinical manifestations range from an asymptomatic presentation to vaginitis, dyspareunia, and strawberry cervix in women and urethritis in men. In addition to these direct symptoms, trichomoniasis also has been associated with premature birth, low infant birth weight, and increased susceptibility to HIV infection. See Cotch, M. F., et al., Sex. Transm. Dis., 24, 353-360 (1997); Sorvillo, F., et al., Lancet, 351, 213-214 (1998).

Metronidazole has been the principal drug prescribed for treatment of trichomoniasis infections since it was introduced in 1960. Durel, P., et al., Br. J. Vener. Dis. 36, 21-26 (1960). Although resistance to metronidazole was first reported in 1962, see Robinson, S. C., Can. Med. Assoc. J., 86, 665 (1962), it is still effective, successfully treating approximately 90-95% of infections. See Centers for Disease Control and Prevention, Sexually Transmitted Diseases Treatment Guidelines. MMWR: Morbidity and Mortality Weekly Report., 42(RR-14), 70-72 (1993). Metronidazole treatment does not cure all patients, however, and recognition of resistance is increasing. Requests to the Centers for Disease Control (CDC) for evaluation of metronidazole resistance in clinical isolates have increased from fifteen in 1995 to over 100 in 2003. In addition, side effects such as gastrointestinal discomfort and nausea are commonly reported. See Smilack, J. D., et al., Mayo Clin. Proc., 66, 1270-1280 (1991). Along with hypersensitivity reactions, these side effects can be severe enough to preclude metronidazole use for treating some individuals. See Kurohara, M. L. et al., J. Allergy Clin. Immunol., 88, 279-280 (1991).

The efficacy of tinidazole against T. vaginalis isolates at lower minimal lethal concentrations (MLCs) than metronidazole has been reported. See Crowell, A. L., et al., Antimicrob. Agents Chemother., 47, 1407-1409 (2003). This finding also is supported by clinical observations. See Sobel, J. D., et al., Clin. Infect. Dis., 33, 1341-1346 (2001). Like metronidazole, tinidazole is a 5-nitroimidazole. Isolates with very high levels of resistance to metronidazole also have increased tinidazole MLCs. See Crowell, A. L., et al., Antimicrob. Agents Chemother., 47, 1407-1409 (2003).

In addition, although tinidazole use results in fewer common side effects than metronidazole, it is possible that persons with hypersensitivity reactions to metronidazole also can have adverse reactions to tinidazole. Taken together, although tinidazole might prove to be useful in many cases of metronidazole treatment failure, identification of non-nitroimidazole compounds that have efficacy against trichomonads is desirable. Accordingly, the presently disclosed subject matter provides dicationic, aromatic diamidine compounds that exhibit activity against Trichomonas vaginalis.

### SUMMARY

In some embodiments, the presently disclosed subject matter provides a method of treating a trichomoniasis infection in a subject in need thereof, the method comprising administering to the subject an effective amount of a compound of Formula (I):
A₁-Ar₁-L-Ar₂-A₂ (I)

wherein:
Ar₁ and Ar₂ are each independently selected from the group consisting of:

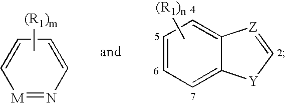

wherein:
M, N, and Z are each independently selected from the group consisting of N and CH;
Y is selected from the group consisting of NR₃, O, S, Se, and Te, wherein R₃ is selected from the group consisting of H, alkyl, and substituted alkyl;
each m is independently an integer from 0 to 2;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl; and

wherein if Ar₁ or Ar₂ is:

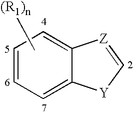

Ar₁ or Ar₂ is attached to L through a bond at carbon 2;
L is selected from the group consisting of:

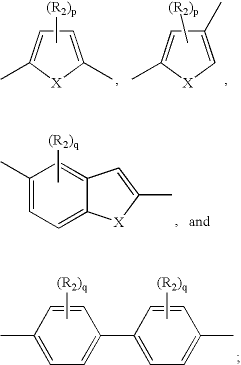

wherein:
p is an integer from 0 to 2;
each q is independently an integer from 0 to 4;
X is selected from the group consisting of O, S, NR₄, Se, and Te, wherein R₄ is selected from the group consisting of H, alkyl, and substituted alkyl;
each R₂ is independently selected from the group consisting of alkyl, substituted alkyl, halo, hydroxyl, alkoxyl, aryl, substituted aryl, aryloxyl, and aralkyoxyl; and
A₁ and A₂ are each independently selected from the group consisting of: 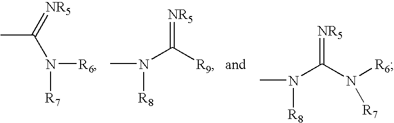
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆ together represent a C₂ to C₁₀ alkyl, C₂ to C₁₀ hydroxyalkyl, or C₂ to C₁₀ alkylene;
or a phamaceutically acceptable salt thereof.

In some embodiments, the presently disclosed subject matter provides compounds of

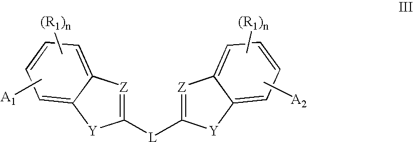

wherein:
Y is selected from the group consisting of NR₃, O, S, Se, and Te, wherein R₃ is selected from the group consisting of H, alkyl and substituted alkyl;
Z is selected from the group consisting of CH and N;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 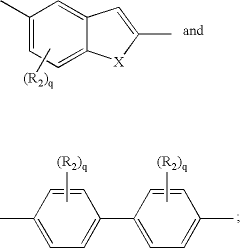
wherein:
   X is selected from the group consisting of O, S, NR₄, Se, and Te, wherein R₄ is selected from the group consisting of H, alkyl, and substituted alkyl;
   q is independently an integer from 0 to 4;
   each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 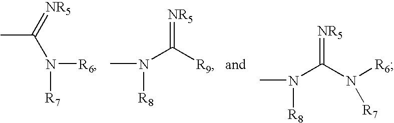
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆ together represent a C₂ to C₁₀ alkyl, C₂ to C₁₀ hydroxyalkyl, or C₂ to C₁₀ alkylene;
or a pharmaceutically acceptable salt thereof.

In some embodiments, the presently disclosed subject matter provides pharmaceutical formulations comprising a compound of Formula (III) in a pharmaceutically acceptable carrier.

In some embodiments, the presently disclosed subject matter provides a process for synthesizing compounds of

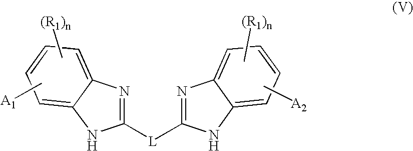

wherein:
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 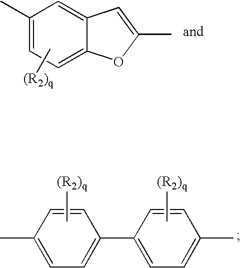
wherein each q is independently an integer from 0 to 4 and each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 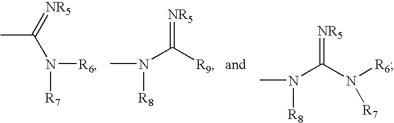
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆ together represent a C₂ to C₁₀ alkyl, C₂ to C₁₀ hydroxyalkyl, or C₂ to C₁₀ alkylene;
or a pharmaceutically acceptable salt thereof;

wherein the process comprises refluxing a mixture of a dialdehyde, two molar equivalents of a diamine and two molar equivalents of an aromatixing reagent in a polar, protic solvent.

It is accordingly an aspect of the presently disclosed subject matter to provide methods and compositions for treating an infection caused by the protozoan parasite Trichomonas vaginalis in a subject in need therof. It is another aspect of the presently disclosed subject matter to provide a process for synthesizing compounds for treating Trichomonas vaginalis infections.

Certain aspects of the presently disclosed subject matter having been stated hereinabove, which are addressed in whole or in part by the presently disclosed subject matter, other aspects will become evident as the description proceeds when taken in connection with the accompanying Examples as best described herein below.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

II. Definitions

Unless otherwise defined, all technical and scientific terms used herein have the same meaning as commonly understood by one of ordinary skill in the art to which this presently described subject matter belongs. All publications, patent applications, patents and other references mentioned herein are incorporated by reference in their entirety.

Throughout the specification and claims, a given chemical formula or name shall encompass all optical and stereoisomers, as well as racemic mixtures where such isomers and mixtures exist.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted with one or more alkyl group substituents, which can be the same or different. The term “alkyl group substituent” includes but is not limited to alkyl, halo, aryl, nitro, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Alkyl groups can further be joined to form a cycloalkyl group or a cycloheteroalkyl group. “Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a cycloheteroalkyl group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl. Representative cycloheteroalkyl groups include piperidine and morpholine.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted with one or more aryl group substituents, which can be the same or different, wherein “aryl group substituent” includes alkyl, aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, aryl, and aralkyl.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

As used herein, the terms “substituted alkyl” and “substituted aryl” include alkyl and aryl groups, as defined herein, in which one or more atoms or functional groups of the aryl or alkyl group are replaced with another atom or functional group, including for example, halogen, aryl, alkyl, alkoxyl, hydroxyl, nitro, amino, alkylamino, dialkylamino, sulfate, and mercapto.

A structure represented generally by a formula such as: 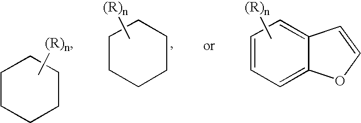
as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure: 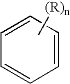
where n is 0 to 2 comprises compound groups including, but not limited to:

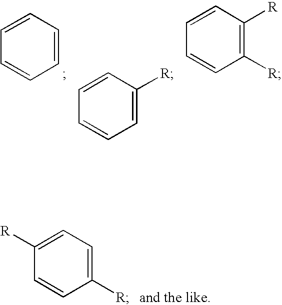

The structure: 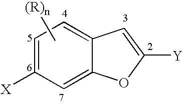
where n is one comprises compound groups including: 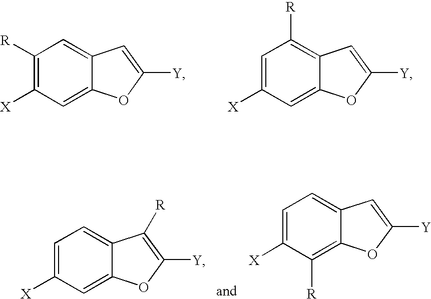
as the one R substituent can be attached at any carbon on the benzofuran parent structure not occupied by another designated substituent, as in this case carbon 6 is substituted by X and carbon 2 is substituted by Y.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

In some embodiments, the compounds described by the presently disclosed subject matter contain a linking group. As used herein, the term “linking group” comprises a chemical moiety, such as a furanyl, phenylene, thienyl, and pyrrolyl radical, which is bonded to two or more other chemical moieties, in particular aryl groups, to form a stable structure.

When a named atom of an aromatic ring or a heterocyclic aromatic ring is defined as being “absent,” the named atom is replaced by a direct bond. When the linking group or spacer group is defined as being absent, the linking group or spacer group is replaced by a direct bond. When a named substituent group, such as an aryl group or a substituted aryl group is defined as being absent, the named substituent group is replaced by a H.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group can be also optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include, but are not limited to, methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Aryloxyl” refers to an aryl-O— group wherein the aryl group is as previously described. The term “aryloxyl” as used herein can refer to phenyloxyl or napthyloxy, and alkyl, halo, or alkoxyl substituted phenyloxyl or napthyloxy.

“Aralkyl” refers to an aryl-alkyl- group wherein aryl and alkyl are as previously described. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl. “Dialkylamino” refers to an —NRR′ group wherein each of R and R′ is independently an alkyl group as previously described. Exemplary alkylamino groups include ethylmethylamino, dimethylamino, and diethylamino.

“Alkoxycarbonyl” refers to an alkyl-O-CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O-CO-group. Exemplary aryloxycarbonyl groups include phenoxy- and naphthoxy-carbonyl.

“Aralkoxycarbonyl” refers to an aralkyl-O-CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described.

“Dialkylcarbamoyl” refers to a R′RN—CO— group wherein each of R and R′ is independently alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term “metal alkyl” refers to a compound of the general formula MRₙ, wherein M is a metal atom, including, but not limited to aluminum, boron, magnesium, zinc, gallium, indium, antimony and related metals, R is an alkyl group as defined herein, and n is an integer. A representative metal alkyl is trimethylaluminum, abbreviated as Al(CH₃)₃ or AlMe₃.

The term “alkali metal alcoholate” refers to an alkali metal derivative of an alcohol having the general formula MₐORₙ, wherein Mₐ is an alkali metal, such as lithium, sodium, potassium, O is oxygen, R is an alkyl group as defined herein, and n is an integer. Representative alkali metal alcoholates include, but are not limited to sodium methanolate, abbreviated as NaOCH₃ or NaOMe, and potassium butoxide, abbreviated as KOC(CH₃)₃.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, etc.

A named “R”, “R₁,” “X,” “Y,” “M,” “N,” “A,” “A₁”, “Ar,” “L,” or “Z” group will generally have the structure that is recognized in the art as corresponding to a group having that name, unless specified otherwise herein. For the purposes of illustration, certain representative “R,” “X,” “Y”, “Ar”, “Z,” “M,” “N,” and “A” groups as set forth above are defined below. These definitions are intended to supplement and illustrate, not preclude, the definitions that would be apparent to one of ordinary skill in the art upon review of the present disclosure.

The term “aprotic solvent” refers to a solvent molecule which can neither accept nor donate a proton. Typical aprotic solvents include, but are not limited to, acetone, acetonitrile, benzene, butanone, butyronitrile, carbon tetrachloride, chlorobenzene, chloroform, 1,2-dichloroethane, dichloromethane, diethyl ether, dimethylacetamide, N,N-dimethylformamide (DMF), dimethylsulfoxide (DMSO), 1,4-dioxane, ethyl acetate, ethylene glycol dimethyl ether, hexane, N-methylpyrrolidone, pyridine, tetrahydrofuran (THF), and toluene. Certain aprotic solvents are polar solvents. Examples of polar aprotic solvents include, but are not limited to, acetone, acetonitrile, butanone, N,N-dimethylformamide, and dimethylsulfoxide. Certain aprotic solvents are non-polar solvents. Examples of nonpolar, aprotic solvents include, but are not limited to, diethyl ether, aliphatic hydrocarbons, such as hexane, aromatic hydrocarbons, such as benzene and toluene, and symmetrical halogenated hydrocarbons, such as carbon tetrachloride.

The term “protic solvent” refers to a solvent molecule which contains a hydrogen atom bonded to an electronegative atom, such as an oxygen atom or a nitrogen atom. Typical protic solvents include, but are not limited to, carboxylic acids, such as acetic acid, alcohols, such as methanol and ethanol, amines, amides, and water.

The term “reflux” and grammatical derivations thereof refer to boiling a liquid, such as a solvent, in a container, such as a reaction flask, with which a condenser is associated, thereby facilitating continuous boiling without loss of liquid, due to the condensation of vapors on the interior walls of the condenser.

II. Methods of Treating Trichomoniasis Infections

Subjects with trichomoniasis infections can be treated by methods described herein. These infections can be caused by the protozoan parasite Trichomonas vaginalis. The methods of the presently disclosed subject matter are useful for treating these conditions in that they inhibit the onset, growth, or spread of the condition, cause regression of the condition, cure the condition, or otherwise improve the general well-being of a subject afflicted with, or at risk of, contracting the condition.

The methods of treating a trichomoniasis infection comprise administering to a subject in need of treatment thereof an active compound as described herein. These active compounds, as set forth above, include compounds of Formula (I), their corresponding prodrugs, and pharmaceutically acceptable salts of the compounds and prodrugs.

With regard to the presently described method embodiments, compounds of Formula (I) can have a structure as follows:
A₁-Ar₁-L-Ar₂-A₂ (I)

wherein:
Ar₁ and Ar₂ are each independently selected from the group consisting of: 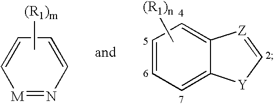
wherein:
   M, N and Z are independently selected from the group consisting of N and CH;
   Y is selected from the group consisting of NR₃, O, S, Se, and Te, wherein R₃ is selected from the group consisting of H, alkyl, and substituted alkyl;
   each m is independently an integer from 0 to 2;
   each n is independently an integer from 0 to 3;
   each R₁ is independently selected from the group consisting of alkyl, substituted alkyl, halo, hydroxyl, alkoxyl, aryl, substituted aryl, aryloxyl, and aralkyloxyl; and
   wherein if Ar₁ or Ar₂ is: 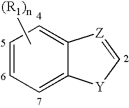
   Ar₁ or Ar₂ is attached to L through a bond at carbon 2; L is selected from the group consisting of: 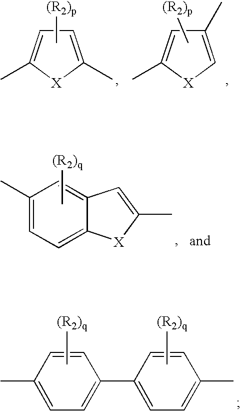
wherein:
   p is an integer from 0 to 2;
   each q is independently an integer from 0 to 4;
   X is selected from the group consisting of O, S, NR₄, Se, and Te, wherein R₄ is selected from the group consisting of H, alkyl, and substituted alkyl;
   each R₂ is independently selected from the group consisting of alkyl, substituted alkyl, halo, hydroxyl, alkoxyl, aryl, substituted aryl, aryloxyl, and aralkyoxyl; and
A₁ and A₂ are each independently selected from the group consisting of: 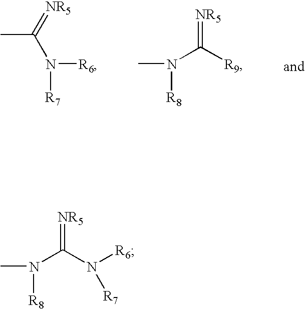
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆ together represent a C₂ to C₁₀ alkyl, C₂ to C₁₀ hydroxyalkyl, or C₂ to C₁₀ alkylene;
or a pharmaceutically acceptable salt thereof.

With regard to the presently described method embodiments, both Ar₁ and Ar₂ of Formula (I) can be monocyclic aromatic groups so that the compounds can be further defined as having a structure of Formula (II) as follows:

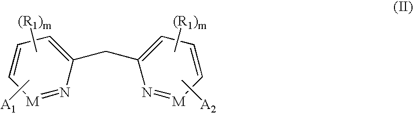

wherein:
each M and N is independently selected from the group consisting of N and CH;
each m is independently an integer from 0 to 2;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 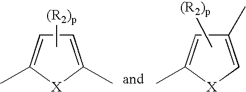
wherein:
   p is an integer from 0 to 2;
   each R₂ is independently selected from the group consisting of alkyl, substituted alkyl, halo, hydroxyl, alkoxyl, aryl, substituted aryl, aryloxyl, and aralkyloxyl;
   X is selected from the group consisting of O, S, NR₄, Se and Te, wherein R₄ is selected from the group consisting of H, alkyl, and substituted alkyl; and
A₁ and A₂ are each independently selected from the group consisting of:

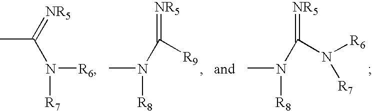

wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;

or a pharmaceutically acceptable salt thereof.

In some embodiments M and N each comprise CH.

In some embodiments L comprises:

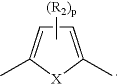

In some embodiments L comprises:

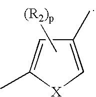

In some embodiments, X is oxygen.

In some embodiments, A₁ and A₂ comprise: 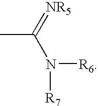
wherein R₆ and R₇ are selected from the group consisting of H, alkyl, substituted alkyl, and cycloalkyl; and R₅ is selected from the group consisting of H, hydroxyl, and alkoxyl. In some embodiments, R₅ is H. In some embodiments, R₆ comprises alkyl. In some embodiments, R₅ comprises alkoxyl.

In some embodiments, A₁ and A₂ comprise:

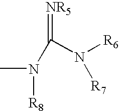

wherein R₅, R₆, R₇, and R₈ are each H.

In some embodiments, the compound of Formula (II) is selected from the group consisting of: 2,5-Bis(4-amidinophenyl)furan (also known as furamidine), 2,5-Bis[4-(O-methyloxyamidino)phenyl]furan, 2,5-Bis[4-(N-isopropylamidino)phenyl]furan, 2,5-Bis[4-(N-cyclohexylamidino)phenyl]-furan, 2,5-Bis-(4-guanidinophenyl)furan and 3,5-Bis(4-amidophenyl)furan. These compounds are shown in Scheme 1 below as 1, 2, 3, 4, 5 and 6 respectively.

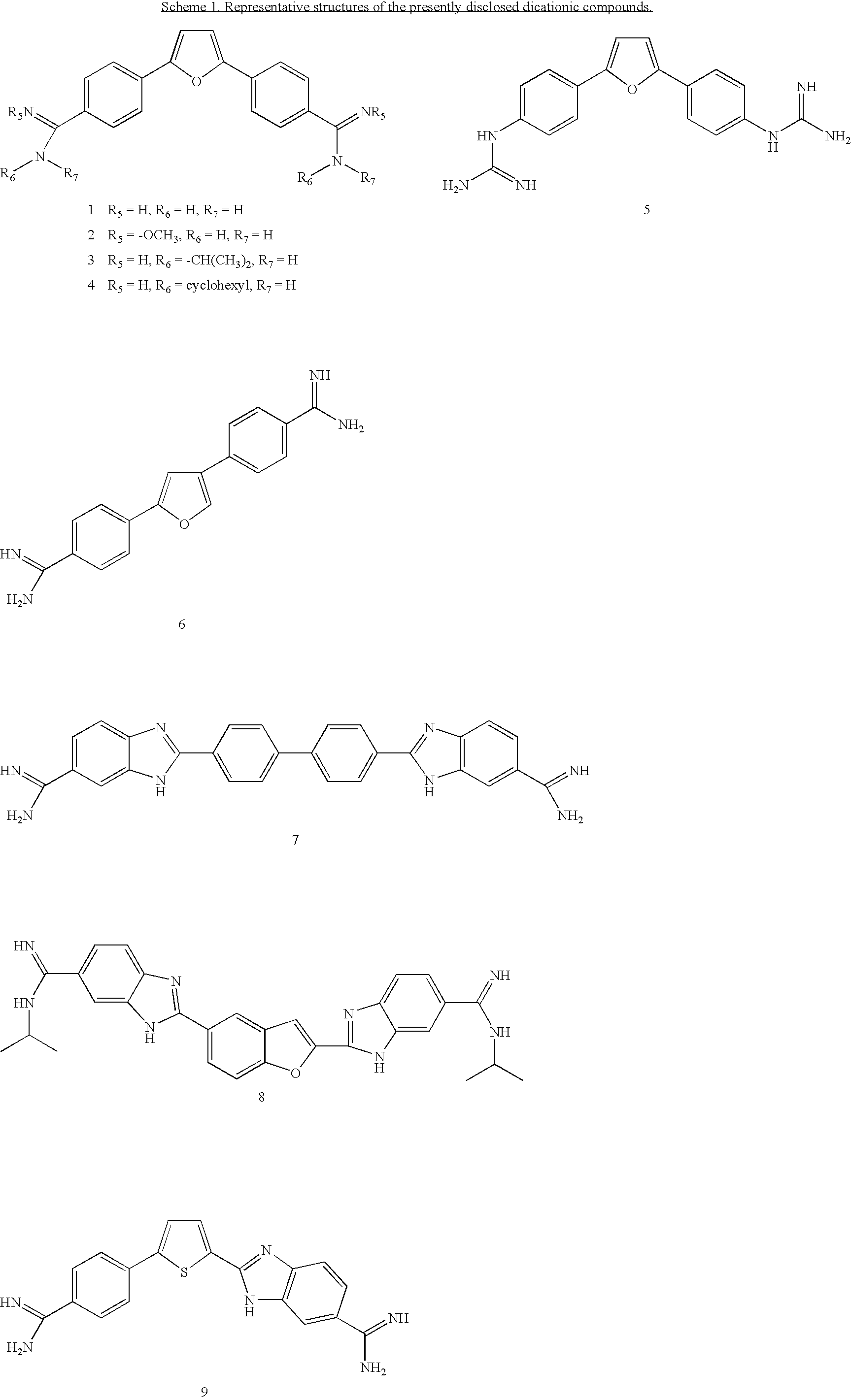

With regard to the presently described method embodiments, Ar₁ and Ar₂ of Formula (I) can each be fused bicyclic aromatic groups, and the compounds can be further defined as having a structure of Formula (III) as follows:

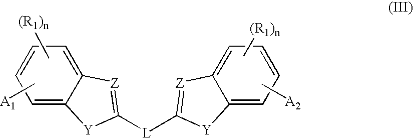

wherein:
Y is selected from the group consisting of NR₃, O, S, Se, and Te, wherein R₃ is selected from the group consisting of H, alkyl, and substituted alkyl;
Z is selected from the group consisting of CH and N;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 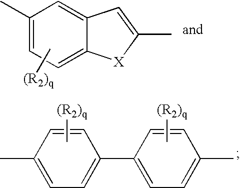
wherein:
   X is selected from the group consisting of O, S, NR₄, Se, and Te, wherein R₄ is selected from the group consisting of H, alkyl and substituted alkyl;
   each q is independently an integer from 0 to 4;
   each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 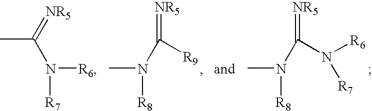
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆ together represent a C₂ to C₁₀ alkyl, C₂ to C₁₀ hydroxyalkyl, or C₂ to C₁₀ alkylene;
or a pharmaceutically acceptable salt thereof.

In some embodiments, Z is N and Y is NH.

In some embodiments, L comprises:

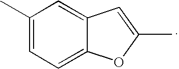

In some embodiments, L comprises:

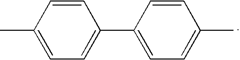

In some embodiments, A₁ and A₂ each comprise: 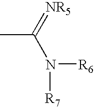
wherein R₆ and R₇ are independently selected from the group consisting of H, alkyl, substituted alkyl, and cycloalkyl; and R₅ is selected from the group consisting of H, hydroxyl, and alkoxyl. In some embodiments, R₅ is H. In some embodiments, R₆ comprises alkyl.

In some embodiments, the compound of Formula (III) is selected from the group consisting of: 4,4′-Bis{2-[(4-amidino) benzimidazolyl]}biphenyl, and 2,5-Bis{2-[5-(N-isopropylamidino)benzimid-azoyl]}benzo[b]furan. These compounds are structures 7 and 8 in Scheme 1 above.

With regard to the presently described method embodiments, one of Ar₁ and Ar₂ of Formula (I) can be a monocyclic aromatic group and the other Ar group can be a bicyclic aromatic group, such that the compound can be further defined as having a structure of Formula (IV) as follows:

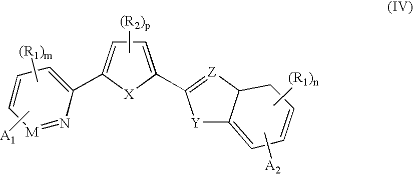

wherein:
M, N and Z are independently selected from the group consisting of N and CH;
Y is selected from the group consisting of NR₃, O, S, Se, and Te, wherein R₃ is selected from the group consisting of H, alkyl, and substituted alkyl;
m is an integer from 0 to 2;
n is an integer from 0 to 3;
p is an integer from 0 to 2;
each R₁ and R₂ is independently selected from the group consisting of alkyl, substituted alkyl, halo, hydroxyl, alkoxyl, aryl, substituted aryl, aryloxyl, and aralkyloxyl;
X is selected from the group consisting of O, S, NR₄, Se, and Te, wherein R₄ is selected from the group consisting of H, alkyl, and substituted alkyl; and
A₁ and A₂ are each independently selected from the group consisting of: 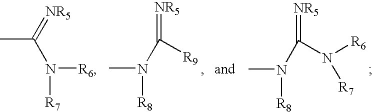
   wherein:
      R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
      R₅ and R₆ together represent a C₂ to C₁₀ alkyl, C₂ to C₁₀ hydroxyalkyl, or C₂ to C₁₀ alkylene;
   or a pharmaceutically acceptable salt thereof.

In some embodiments, M and N are each CH.

In some embodiments, X is sulfur.

In some embodiments, A₁ and A₂ comprise 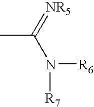
wherein R₅, R₆ and R₇ are each H.

In some embodiments, the compound of Formula (IV) is 2-(4-Amidinophenyl)-5-[2-(5-amidinobenzimidazoyl)]thiophene, compound 9 in Scheme 1 above.

In some embodiments, the trichomoniasis infection is caused by the protozoan parasite Trichomonas vaginalis.

In some embodiments, the compound of Formula (I-IV) is administered to a subject prophylactically to prevent or reduce the incidence of recurrence of the infection.

In some embodiments, the compound of Formula (I-IV) is administered in the form of a pharmaceutically acceptable salt. In some embodiments, the pharmaceutically acceptable salt comprises a hydrochloride salt.

The subject treated in the presently disclosed subject matter in its many embodiments is desirably a human subject, although it is to be understood the methods described herein are effective with respect to all vertebrate species, which are intended to be included in the term “subject.” The methods described herein are particularly useful in the treatment and/or prevention of infectious diseases in warm-blooded vertebrates. Thus, the methods can be used as treatment for mammals and birds.

More particularly, provided herein is the treatment of mammals, such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided herein is the treatment of birds, including the treatment of those kinds of birds that are endangered, kept in zoos or as pets, as well as fowl, and more particularly domesticated fowl, i.e., poultry, such as turkeys, chickens, ducks, geese, guinea fowl, and the like, as they also are of economical importance to humans. Thus, embodiments of the methods described herein include the treatment of livestock, including, but not limited to, domesticated swine (pigs and hogs), ruminants, horses, poultry, and the like.

III. Novel Compounds for Treating Trichomoniasis Infections

A. Novel Compounds of Formula (III)

With regard to the presently described compound embodiments, compounds of Formula (III) are defined as having a structure as follows:

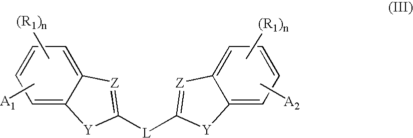

wherein:
Y is selected from the group consisting of NR₃, O, S, Se, and Te, wherein R₃ is selected from the group consisting of H, alkyl, and substituted alkyl;
Z is selected from the group consisting of CH and N;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of:

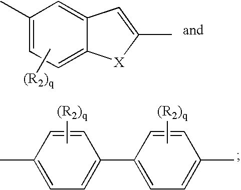

wherein:
X is selected from the group consisting of O, S, NR₄, Se, and Te, wherein R₄ is selected from the group consisting of H, alkyl, and substituted alkyl;
each q is independently an integer from 0 to 4;
each R₂ is independently selected from the group consisting of alkyl, substituted alkyl, halo, hydroxyl, alkoxyl, aryl, substituted aryl, aryloxyl, and aralkyloxyl; and

A₁ and A₂ are each independently selected from the group consisting of:

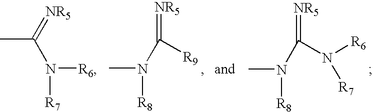

wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;

or a pharmaceutically acceptable salt thereof.

In some embodiments, Z is N and Y is NH.

In some embodiments, L comprises:

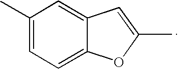

In some embodiments, L comprises:

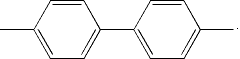

In some embodiments, A₁ and A₂ comprise: 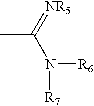
wherein R₆ and R₇ are selected from the group consisting of H, alkyl, substituted alkyl and cycloalkyl; and R₅ is selected from the group consisting of H, hydroxyl, and alkoxyl. In some embodiments, R₅ is H. In some embodiments, R₆ comprises alkyl.

In some embodiments, the compound of Formula (III) is selected from the group consisting of: 4,4′-Bis{2-[(4-amidino)benzimidazolyl]}biphenyl, 7; and 2,5-Bis{2-[5-(N-isopropylamidino)-benzimidazoyl]}benzo[b]furan, 8 as depicted in Scheme 1 above. In some embodiments, the compound of Formula (III) is present as a pharmaceutically acceptable salt. In some embodiments, the compound of Formula (III) is present as a hydrochloride salt.

With regard to the presently described compound embodiments, Y can be NH and Z can be N so that the compounds can be further defined as having a structure of Formula (V) as follows:

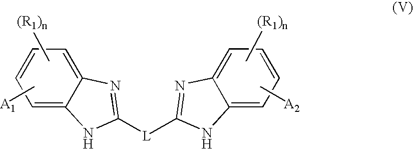

wherein:
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 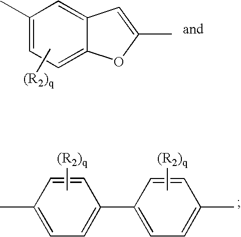
wherein each q is independently an integer from 0 to 4 and each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 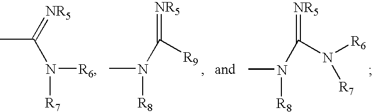
wherein:
   R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
   R₅ and R₆together represent a C₂ to C₁₀ alkyl, C₂ to C₁₀ hydroxyalkyl, or C₂ to C₁₀ alkylene;
or a pharmaceutically acceptable salt thereof.

B. Process for Synthesizing a Compound of Formula (V)

In some embodiments of the presently disclosed subject matter a process for synthesizing a compound of Formula (V) is provided wherein the process comprises refluxing a mixture of a dialdehyde, two molar equivalents of a diamine and two molar equivalents of an aromatizing agent in a polar, protic solvent to form a compound of Formula (V).

In some embodiments, the dialdehyde is selected from the group consisting of 4,4′-diformyl-1,1′-biphenyl and benzo[b]furan-2,5-dicarboxaldehyde.

In some embodiments, the diamine is selected from the group consisting of 4-amidino-1,2-phenylenediamine and 4-N-isopropylamidino-1,2-phenylenediamine.

In some embodiments, the aromatizing agent is 1,4-benzoquinone. In some embodiments, the polar, protic solvent is ethanol.

In some embodiments, the process for synthesizing a compound of Formula (V) further comprises dissolving the compound of Formula (V) in a solvent to form a reaction mixture and treating the reaction mixture with a solvent saturated with HCl to form a hydrochloride salt of the compound of Formula (V).

C. Prodrugs

In representative embodiments, compounds disclosed herein are prodrugs. A prodrug means a compound that, upon administration to a recipient, is capable of providing (directly or indirectly) a compound of the presently disclosed subject matter or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species, for example.

With regard to the presently disclosed compounds, one factor that has been thought to limit the oral bioavailability of similar compounds is the high pKₐ of the amidino or guanidino group. See Ansede, J. H.; et al., J. Med. Chem. 47, 4335-4338 (2004). Prodrug strategies to lower the pKₐ of such groups and provide orally available compounds often include introduction of an oxygen-containing moiety such as hydroxyl or alkoxyl at a nitrogen atom. Thus, some prodrugs for amidines include N-hydroxylated amidines (also known as amidoximes) and N-alkoxylated amidines (also known as O-alkoxyamidines). Compound 2 in Scheme 1 is an amidine prodrug. Such compounds can often be reduced back to the fully active amidine parent drug in vivo or in vitro, for example, by microsomal metabolism.

D. Pharmaceutically Acceptable Salts

Additionally, the active compounds can be administered as pharmaceutically acceptable salts. Such salts include the gluconate, lactate, maleate, acetate, tartarate, citrate, phosphate, borate, nitrate, sulfate, and hydrochloride salts. The salts of the compounds described herein can be prepared, in general, by reacting the base compound with the desired acid in solution. After the reaction is complete, the salts are crystallized from solution by the addition of an appropriate amount of solvent in which the salt is insoluble. In some embodiments, the pharmaceutically acceptable salt is a hydrochloride salt. In other embodiments, the pharmaceutically acceptable salt is an acetate salt.

IV. Pharmaceutical Formulations

The compounds of Formula (I-V), the pharmaceutically acceptable salts thereof, prodrugs corresponding to compounds of Formula (I-V), and the pharmaceutically acceptable salts thereof, are all referred to herein as “active compounds.” Pharmaceutical formulations comprising the aforementioned active compounds also are provided herein. These pharmaceutical formulations comprise active compounds as described herein, in a pharmaceutically acceptable carrier. Pharmaceutical formulations can be prepared for oral, intravenous, or aerosol administration as discussed in greater detail below. Also, the presently disclosed subject matter provides such active compounds that have been lyophilized and that can be reconstituted to form pharmaceutically acceptable formulations for administration, as by intravenous or intramuscular injection.

The therapeutically effective dosage of any specific active compound, the use of which is in the scope of embodiments described herein, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of the infection.

In accordance with the present methods, pharmaceutically active compounds as described herein can be administered orally as a solid or as a liquid, or can be administered intramuscularly or intravenously as a solution, suspension, or emulsion. Alternatively, the compounds or salts also can be administered by inhalation, intravenously, or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise a compound of Formula (I-V) described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. Of course, the dispensing is preferably done by an aseptic method. Sterilized closures can then be placed on the vials and, if desired, the vial contents can be lyophilized.

In addition to compounds of Formula (I-V) or their salts or prodrugs, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain anti-microbial preservatives. Useful anti-microbial preservatives include methylparaben, propylparaben, and benzyl alcohol. The anti-microbial preservative is typically employed when the formulation is placed in a vial designed for multi-dose use. The pharmaceutical formulations described herein can be lyophilized using techniques well known in the art.

In yet another embodiment of the subject matter described herein, there is provided an injectable, stable, sterile formulation comprising a compound of Formula (I-V), or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the compound or salt is substantially water-insoluble, a sufficient amount of emulsifying agent, which is physiologically acceptable, can be employed in sufficient quantity to emulsify the compound or salt in an aqueous carrier. One such useful emulsifying agent is phosphatidyl choline.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations containing the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations car be employed without departing from the scope of the presently disclosed subject matter.

### Materials and Methods Parasite Isolates and Compounds

CDC reference strains 085 and 520 that are metronidazole resistant and sensitive, respectively, were maintained at 37° C. in Diamond's trypticase-yeast-maltose medium (TYM; pH 6.0). Metronidazole, tinidazole, pentamidine, and berenil were purchased from Sigma Chemical Co. (St. Louis, Mo., United States of America). Compound 1, see Das, B. P. and D. W. Boykin, J. Med. Chem., 20, 531-536 (1977); Compound 2, see Boykin, D. W., et al., Bioorganic and Med. Chem. Lett., 6:3017-3020 (1996); Compound 3 and Compound 4, see Boykin, D. W., et al., J. Med. Chem., 41, 124-129 (1998); Compound 6, see Francesconi, I., et al., J. Med. Chem., 42, 2260-2265 (1999); Compound 5, see Stephens, C., et al., J. Med. Chem., 44, 1741-1748 (2001); and Compound 9, see Mallena, S. et al., J. Am. Chem. Soc., 126, 13659-13669 (2004); were synthesized as previously reported. Purity was determined by NMR and TLC. The syntheses of 7 and 8 are outlined below. See also Crowell, A. L., et al., Antimicrob. Agents Chemother., 48, 3602-3605 (2004), which is incorporated herein by reference in its entirety.

4,4′-Bis{2-[(4-amidino)benzimidazolyl]}biphenyl tetrahydrochloride (7). A mixture of 4,4′-diformyl-1,1′-biphenyl (0.21 g, 0.001 mole), 4-amidino-1,2-phenylenediamine hydrochloride hemihydrate (0.39 g, 0.002 mole) and 1,4-benzoquinone (0.216 g, 0.002 mole) in ethanol was heated at reflux for 12 h. The solvent was reduced to one third, followed by dilution with ether and then filtration, yielding a dark solid. The solid was dissolved in a large volume of hot ethanol and filtered; the solution was treated with 10 mL HCl gas saturated ethanol and stirred. The solvent was reduced to one third and diluted with ether. A dark hydrochloride salt precipitated, which was filtered, washed with ether, and dried in vacuum at 75° C. for 24 h to yield 0.43 g (66%); mp>300° C. dec.; ¹H-NMR (DMSO-d₆): 8.35(d, 4H, J=7.6 Hz), 8.21(s, 2H), 8.02(d, 4H, J=7.6 Hz), 7.85(d, 4H, J=8.4 Hz), 7.50(d, 4H, J=8.4 Hz); ¹³C NMR (DMSO-d₆): 166.0, 153.2, 141.4, 137.5, 128.4, 127.8, 126.9, 123.4, 122.6, 116.2, 115.1; FAB MS: m/e 483(M⁺+1); Analysis calculated for C₂₉H₂₂N₈.4HCl.1.5H₂O: C, 53.41; H, 4.46: N, 17.09. Found: C, 52.97; H, 4.61; N, 17.17.

2,5-Bis{2-[5-(N-isopropylamidino)benzimidazoyl]}benzo[b]furan tetrahydrochloride (8). A protocol similar to that described above for 7, involving the condensation of benzo[b]furan-2,5-dicarboxaldehyde and 4-N-isopropylamidino-1,2-phenylenediamine gave a metallic green solid in 69% yield; mp 285°C.-290° C. ¹H NMR (DMSO-d₆/80° C.) 8.71 (s, 1H), 8.36 (d, 1H, J=8.8 Hz) 8.08(d, 2H, J=9.2 Hz), 7.98 (d, 1H, J=8.8 Hz), 7.96 (s, 1H), 7.85 (d, 1H, J=8.8 Hz), 7.82 (d, 1H, J=8.8 Hz), 7.64 (d, 1H, J=8.8 Hz), 7.61 (d, 1H, J=8.8 Hz), 4.02 (broad q, 2H, J=6 Hz), 1.32(broad d, 12H, J=6 Hz). ¹³C NMR (DMSO-d₆/D₂O/80° C.) 162.9, 162.6, 157.5, 152.9, 147.4, 145.3, 141.1, 138.7, 137.8, 134.9, 129.2, 126.9, 125.7, 125.0, 124.5, 123.8, 123.2, 121.4, 116.9, 116.1, 115.7, 115.5, 113.9, 109.4, 46.2, 46.1, 21.6 (signals overlap). Analysis calculated for C₃₀H₃₀N₈O.4HCl.0.5H₂O: C, 53.49; H, 5.23; N, 16.64. Found: C, 53.53; H, 5.29; N, 16.45.

### Assays

Compounds were dissolved in dimethyl sulfoxide (DMSO, Sigma) and further diluted with Diamond's TYM media to reach assay concentrations. Two types of assays were performed on the cationic compounds being evaluated. An initial screen was performed using the standard MLC assay, see Crowell, A. L., et al., Antimicrob. Agents Chemother., 47, 1407-1409 (2003); Meingassner, J. G. and J. Thurner, Antimicrob. Agents Chemother., 15, 254-257 (1979), with a maximum concentration of 20 μM for the test compounds. After 48 h of incubation at 37° C., plates were examined using an inverted phase-contrast microscope. The lowest drug concentration at which no motile trichomonads were observed was recorded as the MLC. Each compound was tested at least twice under both aerobic and anaerobic conditions. Anaerobic conditions were generated using a GASPAK™ jar and CO₂-generating GASPAK™ Plus anaerobic system envelopes (Becton Dickinson, Sparks, Md., United States of America) and monitored with GASPAK™ disposable anaerobic indicator strips (Becton Dickinson). Compounds that showed no activity were not tested further. To further evaluate the compounds' activity, a second type of assay was used to determine the concentration at which 50% of the parasite growth was inhibited (IC₅₀). In these assays, 0.5 μCi of tritiated thymidine (Perkin-Elmer, Boston, Mass., United States of America) was added to each well of a standard assay at the initiation of culture. At 48 h of incubation under either aerobic or anaerobic conditions, cells were harvested onto glass fiber filters (Wallac, Turku, Finland) using a TOMTEC™ cell harvester (Hamden, Conn., United States of America). Incorporated thymidine was detected using BETAPLATE™ (Wallac) scintillation fluid and plate reader. The resulting counts per minute over the compound's concentration range were utilized to calculate IC₅₀s using GRAPHPAD™ PRISM™ (GraphPad Software, Inc., San Diego, Calif., United States of America).

### Results

Structures of the Formula (I-V) dicationic compounds that were tested in the assays described above are shown in Scheme 1. Table 1 contains their in vitro IC₅₀ activities against metronidazole-sensitive and -resistant T. vaginalis isolates as well as comparison data for the nitroimidazoles metronidazole and tinidazole. The evaluation of the classical antiprotozoan dicationic molecules pentamidine and berenil also are included. Interestingly, both of these latter two compounds are not effective against T. vaginalis. In contrast, furamidine, 1, the parent molecule in the 2,5-diphenylfuran family of diamidines, shows good activity that is comparable to that of metronidazole against 520, the metronidazole sensitive isolate. It is clearly more effective than either metronidazole or tinidazole against the resistant strain 085 under aerobic conditions. These data suggest, as expected based on structure, a different mode of action for 1 as compared to the nitroimidazoles. The N-alkyl analogs of furamidine, 3 and 4, show similar in vitro effectiveness. Interestingly, 5, a guanidino analog of 1, and 6, the 2,4-diphenylfuran isomer of 1, are not effective against T. vaginalis in these assays. Thus, the 2,5-diphenyl furan family of dications is quite effective versus T. vaginalis in vitro; but the activity appears to be sensitive to structure, as demonstrated by the lack of activity of 5 and 6 in these assays.

A prodrug of furamidine, compound 2, was generally ineffective in vitro as the biochemical pathways necessary to convert it to the active form were not present. Compound 2, however, can be administered orally to provide systemic, efficacious concentrations of 1 and is currently in phase 11 trials for treatment of African trypansomiasis, having successfully completed phase I clinical trials. See Tidwell, R. R., and D. W. Boykin, Dicationic DNA Minor Groove Binders as Antimicrobial Agents, in Small Molecule DNA and RNA Binders: From Synthesis to Nucleic Acid Complexes, vol. 2, (M. Demeunynck, C. Bailly, and W. D. Wilson, ed., Wiley-VCH, New York, 2003) p. 416-460.

The bis-benzimidazoles 7 and 8 also are effective anti-trichomonads. Interestingly, 7 has activity against the metronidazole-resistant 085 strain, but not against the metronidazole-sensitive 520 isolate. Thus, 7 could be a useful tool to evaluate the biochemical basis of T. vaginalis resistance to metronidazole. The most effective dication evaluated herein is the mono-benzimidazole 9. This compound demonstrated IC₅₀ values of 1 μMol or less for both metronidazole sensitive and resistant isolates under either aerobic or anaerobic conditions. The mono-benzimidazole 9 has been successfully used in vivo to treat a different protozoal infection in an experimental model without overt evidence of toxicity to the host.

TABLE 1
IC₅₀ values (μM ± SEM) against metronidazole-sensitive and
metronidazole-resistan T. vaginalis.

| | 085 aerobic | | 085 anaerobic | | 520 aerobic | | 520 anaerobic | |
|---|---|---|---|---|---|---|---|---|
| Compound | n | | n | | n | | n | |
| Metronidazole | 61 | 302.6 ± 22.2 | 62 | 12.3 ± 23.8 | 38 | 18.2 ± 4.25 | 31 | 1.89 ± 0.77 |
| Tinidazole | 5 | 45.1 ± 5.38 | 4 | 4.81 ± 1.17 | 2 | 1.48 ± 0.12 | 2 | 0.004 ± 0.0 |
| Pentamidine | 3 | no effect | 3 | no effect | 3 | no effect | 3 | no effect |
| Berenil | 3 | no effect | 3 | no effect | 3 | no effect | 3 | no effect |
| 1 | 8 | 8.12 ± 2.45 | 7 | 18.6 ± 19.8 | 4 | 18.6 ± 6.43 | 4 | 57.9 ± 19.6 |
| 2 | 3 | 39.1 ± 25.1 | 2 | no effect | 2 | no effect | 2 | no effect |
| 3 | 7 | 6.44 ± 3.89 | 7 | 9.41 ± 7.92 | 7 | 6.60 ± 1.76 | 7 | 3.91 ± 1.06 |
| 4 | 6 | 22.4 ± 13.7 | 6 | 10.2 ± 8.47 | 2 | 15.9 ± 0.74 | 3 | 13.9 ± 3.74 |
| 8 | 6 | 7.27 ± 3.76 | 5 | 37.6 ± 43.9 | 2 | 44.7 ± 9.33 | 3 | 143.1 ± 127 |
| 7 | 8 | 7.79 ± 4.20 | 6 | 25.3 ± 43.1 | 2 | no effect | 2 | no effect |
| 6 | 7 | no effect | 6 | no effect | 3 | no effect | 3 | no effect |
| 5 | 7 | no effect | 6 | no effect | 3 | no effect | 3 | no effect |
| 9 | 7 | 0.27 ± 0.04 | 7 | 0.50 ± 0.42 | 6 | 0.98 ± 0.19 | 6 | 1.24 ± 0.39 |

085 = metronidazole-resistant strain of T. vaginalis.
520 = metronidazole-sensitive strain of T. vaginalis.

### References

The references listed below as well as all references cited in the specification are incorporated herein by reference to the extent that they supplement, explain, provide a background for or teach methodology, techniques and/or compositions employed herein.
Ansede, J. H., et al., J. Med. Chem., 47, 4335-4338 (2004).
Boykin, D. W., et al., Bioorganic and Med. Chem. Let., 6, 3017-3020 1996).
Boykin, D. W., et al., J. Med. Chem., 41, 124-129 (1998).
Centers for Disease Control and Prevention, MMWR: Morbidity and Mortality Weekly Report, 42(RR-14), 70-72 (1993).
Chavalitshewinkoon-Petmitr, P., et al., J. Antimicrob. Chemother., 52, 287-289 (2003).
Cotch, M. F., et al., Sex. Transm. Dis. 24:353-360 (1997).
Crowell, A. L., et al., Antimicrob. Agents Chemother., 48, 3602-3605 (2003).
Crowell, A. L., et al., Antimicrob. Agents Chemother., 47, 1407-1409 (2004).
Das, B. P., and D. W. Boykin. J. Med. Chem., 20, 531-536. (1977).
Durel, P., et al., Br. J. Vener. Dis. 36, 21-26 (1960).
Francesconi, I., et al., J. Med. Chem. 42, 2260-2265 (1999).
Kurohara, M. L., et al., J. Allergy Clin. Immunol. 88, 279-280 (1991).
Mallena, S., et al., J. Am. Chem. Soc., 126, 13659-13669 (2004).
Meingassner, J. G. and J. Thurner, Antimicrob. Agents Chemother., 15, 254-257 (1979).
Robinson, S. C. Can. Med. Assoc. J., 86, 665 (1962).
Smilack, J. D., et al., Mayo Clin. Proc., 66, 1270-1280 (1991).
Sobel, J. D., et al., Clin. Infect. Dis., 33, 1341-1346 (2001).
Sorvillo, F. and P. Kerndt, Lancet., 351, 213-214 (1998).
Stephens, C. E., et al., J. Med. Chem., 44, 1741-1748 (2001).
Tidwell, R. R., and D. W. Boykin, Dicationic DNA Minor Groove Binders as Antimicrobial Agents in Small Molecule DNA and RNA Binders:

From Synthesis to Nucleic Acid Complexes, vol 2. (M. Demeunynck, C. Bailly, and W. D. Wilson, ed., Wiley-VCH, New York, 2003), 416-460.
U.S. Pat. No. 5,628,984.
World Health Organization, An Overview of Selected Curable Sexually Transmitted Diseases In Global Program on AIDS. (World Health Organization, Geneva, Switzerland, 1995), 2-27.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method of treating a trichomoniasis infection in a subject in need thereof, the method comprising administering to the subject an effective amount of a compound of Formula (I):
A₁-Ar₁-L-Ar₂-A₂ (I)
wherein:
Ar₁ and Ar₂ are each independently selected from the group consisting of: 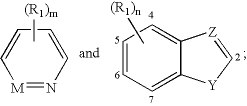
wherein:
M, N and Z are each independently selected from the group consisting of N and CH;
Y is selected from the group consisting of NR₃, O, S, Se, and Te, wherein R₃ is selected from the group consisting of H, alkyl, and substituted alkyl;
each m is independently an integer from 0 to 2;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl; and
wherein if Ar₁ or Ar₂ is: 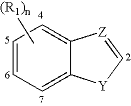
Ar₁ or Ar₂ is attached to L through a bond at carbon 2;
L is selected from the group consisting of: 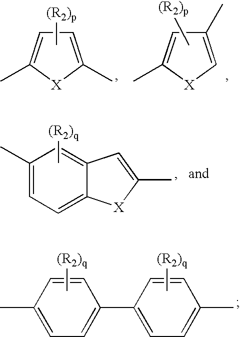
wherein:
p is an integer from 0 to 2;
each q is independently an integer from 0 to 4;
X is selected from the group consisting of O, S, NR₄, Se, and Te, wherein R₄ is selected from the group consisting of H, alkyl, and substituted alkyl;
each R₂ is independently selected from the group consisting of alkyl, substituted alkyl, halo, hydroxyl, alkoxyl, aryl, substituted aryl, aryloxyl, and aralkyoxyl; and
A₁ and A₂ are each independently selected from the group consisting of: 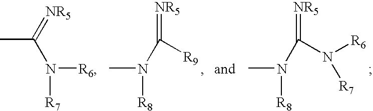
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;
or a pharmaceutically acceptable salt thereof.

2. The method of claim 1, wherein the compound of Formula (I) comprises a compound of Formula (II): 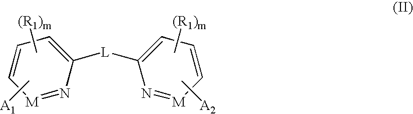
wherein:
each M and N is independently selected from the group consisting of N and CH;
each m is independently an integer from 0 to 2;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 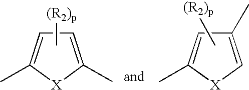
wherein:
p is an integer from 0 to 2;
each R₂ is independently selected from the group consisting of alkyl, substituted alkyl, halo, hydroxyl, alkoxyl, aryl, substituted aryl, aryloxyl, and aralkyloxyl;
X is selected from the group consisting of O, S, NR₄, Se, and Te, wherein R₄ is selected from the group consisting of H, alkyl, and substituted alkyl; and
A₁ and A₂ are each independently selected from the group consisting of: 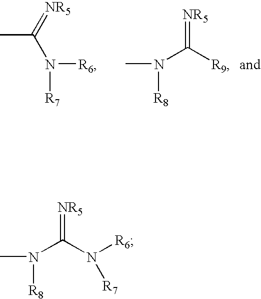
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;
or a pharmaceutically acceptable salt thereof.

3. The method of claim 2, wherein M and N are each CH.

4. The method of claim 2, wherein L comprises:

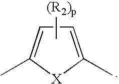

5. The method of claim 2, wherein L comprises:

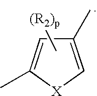

6. The method of claim 2, wherein X is oxygen.

7. The method of claim 2, wherein A₁ and A₂ each comprise: 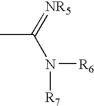
and wherein R₆ and R₇ are independently selected from the group consisting of H, alkyl, substituted alkyl, and cycloalkyl; and R₅ is selected from the group consisting of H, hydroxyl, and alkoxyl.

8. The method of claim 2, wherein A₁ and A₂ each comprise: 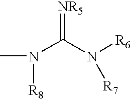
and wherein R₅, R₆, R₇, and R₈ are each H.

9. The method of claim 2, wherein the compound is selected from the group consisting of:
2,5-Bis(4-amidinophenyl)furan;
2,5-Bis[4-(O-methyloxyamidino)phenyl]furan;
2,5-Bis[4-(N-isopropylamidino)phenyl]furan;
2,5-Bis[4-(N-cyclohexylamidino)phenyl]furan;
2,5-Bis(4-guanidinophenyl)furan; and
3,5-Bis(4-amidinophenyl)furan.

10. The method of claim 1, wherein the compound of Formula (I) comprises a compound of Formula (III): 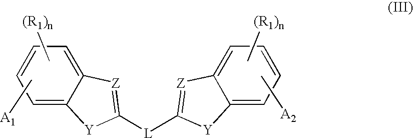
wherein:
Y is selected from the group consisting of NR₃, O, S, Se, and Te, wherein R₃ is selected from the group consisting of H, alkyl, and substituted alkyl;
Z is selected from the group consisting of CH and N;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 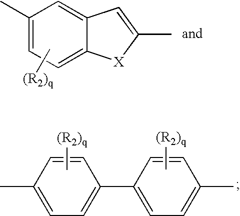
wherein:
X is selected from the group consisting of O, S, NR₄, Se, and Te, wherein R₄ is selected from the group consisting of H, alkyl, and substituted alkyl;
each q is independently an integer from 0 to 4;
each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 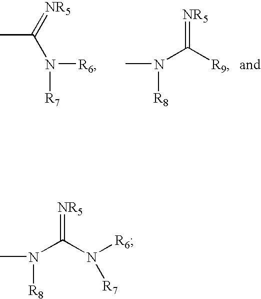
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;
or a pharmaceutically acceptable salt thereof.

11. The method of claim 10, wherein Y is NH and Z is N.

12. The method of claim 10, wherein L comprises:

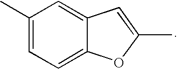

13. The method of claim 10, wherein L comprises:

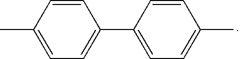

14. The method of claim 10, wherein each A₁ and A₂ comprise 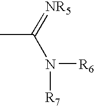
and wherein R₆ and R₇ are independently selected from the group consisting of H, alkyl, substituted alkyl, and cycloalkyl; and R₅ is selected from the group consisting of H, hydroxyl, and alkoxyl.

15. The method of claim 10, wherein the compound is selected from the group consisting of 4,4′-Bis{2-[(4-amidino)benzimidazoyl]}biphenyl and 2,5-Bis{2-[5-(N-isopropylamidino)benzimidazoyl]}benzo[b]furan.

16. The method of claim 1, wherein the compound of Formula (I) comprises a compound of Formula (IV): 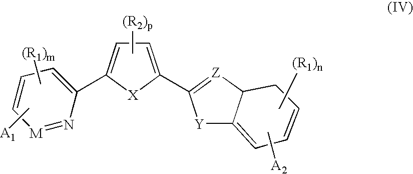
wherein:
M, N and Z are each independently selected from the group consisting of N and CH;
Y is selected from the group consisting of NR₃, O, S, Se, and Te, wherein R₃ is selected from the group consisting of H, alkyl, and substituted alkyl;
m is an integer from 0 to 2;
n is an integer from 0 to 3;
p is an integer from 0 to 2;
each R₁ and R₂ is independently selected from the group consisting of alkyl, substituted alkyl, halo, hydroxyl, alkoxyl, aryl, substituted aryl, aryloxyl, and aralkyloxyl;
X is selected from the group consisting of O, S, NR₄, Se, and Te, wherein R₄ is selected from the group consisting of H, alkyl, and substituted alkyl; and
A₁ and A₂ are each independently selected from the group consisting of: 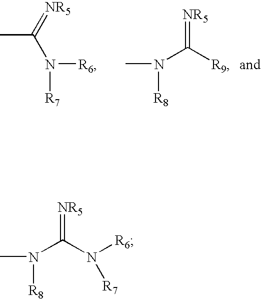
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆together represent a C₂ to C₁₀ alkyl, C₂ to C₁₀ hydroxyalkyl, or C₂ to C₁₀ alkylene;
or a pharmaceutically acceptable salt thereof.

17. The method of claim 16, wherein M and N are each CH.

18. The method of claim 16, wherein Y is NH and Z is N.

19. The method of claim 16, wherein X is sulfur.

20. The method of claim 16, wherein A₁ and A₂ each comprise: 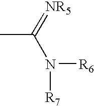
wherein R₅, R₆ and R₇ are each H.

21. The method of claim 16, wherein the compound is 2-(4-Amidinophenyl)-5-[2-(5-amidinobenzimidazoyl)]thiophene.

22. The method of claim 1, wherein the trichomoniasis infection is caused by the protozoan parasite Trichomonas vaginalis.

23. The method of claim 1, wherein the compound of Formula (I) comprises a prodrug.

24. The method of claim 1, wherein the compound of Formula (I) is administered in the form of a pharmaceutically acceptable salt.

25. The method of claim 24, wherein the pharmaceutically acceptable salt comprises a hydrochloride salt.

26. The method of claim 1, wherein the subject is a human.

27. The method of claim 1, comprising administering the compound of Formula (I) orally in one of a solid or a liquid formulation.

28. The method of claim 1, comprising administering the compound in a liposomal formulation.

29. The method of claim 1, comprising administering the compound of Formula (I) to prevent or reduce the incidence of recurrence of the T. vaginalis infection.

30. A compound of Formula (III): 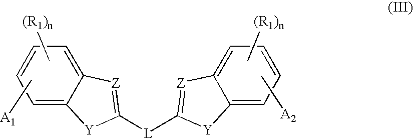
wherein:
Y is selected from the group consisting of NR₃, O, S, Se, and Te, wherein R₃ is selected from the group consisting of H, alkyl, and substituted alkyl;
Z is selected from the group consisting of CH and N;
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 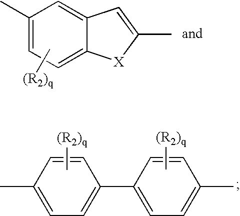
wherein:
X is selected from the group consisting of O, S, NR₄, Se, and Te, wherein R₄ is selected from the group consisting of H, alkyl, and substituted alkyl;
each q is independently an integer from 0 to 4;
each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 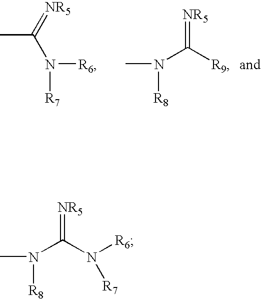
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;
or a pharmaceutically acceptable salt thereof.

31. The compound of claim 30, wherein Z is N and Y is NH.

32. The compound of claim 30, wherein L comprises:

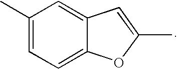

33. The compound of claim 30, wherein L comprises:

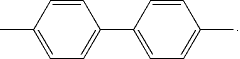

34. The compound of claim 30 wherein A₁ and A₂ each comprise: 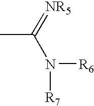
wherein R₆ and R₇ are independently selected from the group consisting of H, alkyl, substituted alkyl and cycloalkyl; and R₅ is selected from the group consisting of H, hydroxyl, and alkoxyl.

35. The compound of claim 30, wherein the compound is selected from the group consisting of 4,4′-Bis{2-[(4-amidino)benzimidazoyl]}biphenyl, 2,5-Bis{2-[5-(N-isopropylamidino)benzimidazoyl]}benzo[b]furan, and pharmaceutically acceptable salts thereof,

36. A compound of claim 30, wherein the pharmaceutically acceptable salt is a hydrochloride salt.

37. A pharmaceutical formulation comprising:
(a) a compound of Formula (III); and
(b) a pharmaceutically acceptable carrier.

38. A method of preparing a compound of Formula (V): 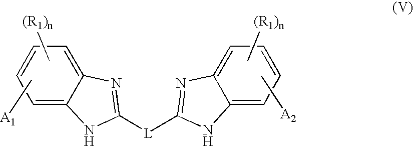
wherein:
each n is independently an integer from 0 to 3;
each R₁ is independently selected from the group consisting of alkyl, substituted alkyl, halo, hydroxyl, alkoxyl, aryl, substituted aryl, aryloxyl, and aralkyloxyl;
L is selected from the group consisting of: 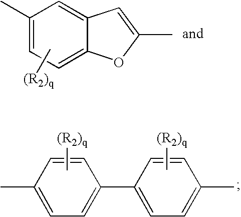
wherein each q is independently an integer from 0 to 4 and each R₂ is independently selected from the group consisting of alkyl, substituted alkyl, halo, hydroxyl, alkoxyl, aryl, substituted aryl, aryloxyl, and aralkyloxyl; and
A₁ and A₂ are each independently selected from the group consisting of: 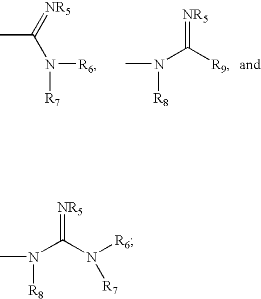
wherein:
R₅, R₆, R₇, R₈, and R₉ are each independently selected from the group consisting of H, alkyl, substituted alkyl, cycloalkyl, aryl, substituted aryl, aralkyl, hydroxyl, alkoxyl, hydroxyalkyl, hydroxycycloalkyl, alkoxycycloalkyl, aminoalkyl, acyloxyl, alkylaminoalkyl, and alkoxycarbonyl; or
R₅ and R₆ together represent a C₂ to C₁₀ alkyl, C₂ to C₁₀ hydroxyalkyl, or C₂ to C₁₀ alkylene;
the method comprising refluxing a mixture of a dialdehyde, two molar equivalents of a diamine and two molar equivalents of an aromatizing reagent in a polar, protic solvent to form a compound of Formula (V).

39. The method of claim 38, wherein the dialdehyde is selected from the group consisting of 4,4′-diformyl-1,1′-biphenyl and benzo[b]furan-2,5-dicarboxaldehyde.

40. The method of claim 38, wherein the diamine is selected from the group consisting of 4-amidino-1,2-phenylenediamine and 4-N-isopropylamidino-1,2-phenylenediamine.

41. The method of claim 38, wherein the aromatizing reagent comprises 1,4-benzoquinone.

42. The method of claim 38, wherein the polar, protic solvent comprises ethanol.

43. The method of claim 38, comprising:
(a) dissolving the compound of Formula (V) in a solvent to form a reaction mixture; and
(b) treating the reaction mixture with a solvent saturated with HCl to form a hydrochloride salt of the compound of Formula (V).
